(19) United States
(12) Patent Application Publication
(10) Pub. No.: US 2015/0110853 A1
(43) Pub. Date: Apr. 23, 2015

(54) TREATMENT OF RADICAL PROSTATECTOMY-INDUCED ERECTILE DYSFUNCTION

(71) Applicants: Hossein A. Ghanbari, Potomac, MD (US); Zhi-Gang Jiang, Gaithersburg, MD (US)

(72) Inventors: Hossein A. Ghanbari, Potomac, MD (US); Zhi-Gang Jiang, Gaithersburg, MD (US)

(73) Assignee: PANACEA PHARMACEUTICALS, Gaithersburg, MD (US)

(21) Appl. No.: 14/057,366

(22) Filed: Oct. 18, 2013

(51) Int. Cl. C07D 213/73

(52) U.S. Cl. CPC C07D 213/73
USPC 424/450; 514/353

(57) ABSTRACT

The present invention provides methods and compositions preventing and treating radical prostatectomy-induced erectile dysfuntion. One embodiment of the present invention is directed to a method of preventing and treating radical prostatectomy-induced erectile dysfuntion by administering to a patient in need at least one thiosemicarbazone compound.

### BACKGROUND OF THE INVENTION

Prostate cancer is a fatal disease in men. Based on the report of Cancer Research UK, prostate cancer is the second most common cancer in men. An estimated 900,000 men worldwide were diagnosed with prostate cancer in 2008. Prostate cancer is the sixth most common cause of death from cancer in men worldwide, estimated to be responsible for 258,000 deaths in 2008. The American Cancer Society reported that about 2.5 million men in the United States have been diagnosed with prostate cancer by August 2013. In 2013, about 238,590 new cases of prostate cancer will be diagnosed, and about 29,720 men will die of prostate cancer. The numbers of incidence and death for prostate cancer are estimated as the second leading cause of cancer death in American men, behind only lung cancer. About 1 man in 36 will die of prostate cancer.

There are several ways to treat prostate cancer, including surgery, radiotherapy, hormone therapy, chemotherapy, etc. Radical prostatectomy is applied to remove the tumor that is confined in the prostate gland in the patients in clinical T1/T2a-b stages. For the patients with a long life expectancy, radical prostatectomy remains the most effective approach with respect to both oncologic success and maximization of quality of life. The open prostatectomy includes radical retropubic and radical perineal prostatectomy.

Erectile dysfunction is the major discouraging risk for the radical prostatectomy. The cavernous nerves are post-ganglionic parasympathetic nerves that facilitate penile erection. They arise from cell bodies in the inferior hypogastric plexus where they receive the pre-ganglionic pelvic splanchnic nerves (S2-S4). These nerves are susceptible to injury following prostatectomy. To avoid injuring the nerves and causing erectile dysfunction complications, nerve sparing prostatectomy was invented. A small electrical stimulation is applied to the nerve and the erectile function with a penile plethysmograph is measured to identify and avoid the nerves that are difficult to see. The nerve sparing technique used in radical prostatectomy enables a complete cancer resection while maximally preserving urinary and sexual function.

However, even with the most highly developed nerve sparing radical prostatectomy technique, traction, contusion and incision of the cavernous nerve may inadvertently occur at the time of radical prostatectomy. Just the trauma to these nerves itself negatively affects erectile nerve tissue health leading to decreased postoperative erectile responses. Although the erectile function could be recovered in time, the recovery rates for nerve sparing-radical retropubic and nerve sparing-radical perineal prostatectomy by 12 months are only 50% and 54.5%, respectively. The recovery rate of erectile function is maximally 70% within 24 months post nerve sparing-radical perineal prostatectomy. The dysfunctional erection status still lingers as a major postoperative problem.

Oxidative stress, indicated by overproduction of reactive oxygen species and/or suppression of antioxidant systems, seems to play key roles in the postoperative nerve injury. Thioredoxin and glutathione systems are both present in the cells as redox defense systems. In the thioredoxin system, thioredoxin reductase converts oxidized thioredoxin into reduced thioredoxin, which is essential for maintenance of redox balance. Thus, this system provides neuroprotection after injury of peripheral nerves. Thioredoxin reductase 2 knockdown induces a significant increase in hydrogen peroxide (H₂O₂), an end product of reactive oxygen species that cause cell damage. Similar to thioredoxin system, glutathione reductase converts oxidized glutathione into reduced glutathione in glutathione system, which maintains the redox stability in the cells. This system also provides neuroprotection after injury of peripheral nerves. Depletion of glutathione leaves neurons susceptible to deleterious reactive metabolites and apoptosis. Immunophilin ligands FK506 has been found to preserve erectile function following cavernous nerve injury in adult rat model. The neuroprotective effect of FK506 in preserving erectile function after cavernous nerve injury has been demonstrated involving antioxidative nitrosative and antiapoptotic mechanisms by up-regulations of expressions of thioredoxin reductase 2, S-nitrosoglutathione reductase, and anti-apoptotic Bcl-2.

FK506 is being developed in phase II clinical trials for treatment of prostatectomy-induced erectile dysfunction. However, FK506 is an immunosuppressive agent. Although FK506 has not been found to stimulate the growth of cancer cells, it should be very cautious to use it as neuroprotectant in cancer patient. There still is a need for methods to properly treat radical prostatectomy-induced ED, and these neuroprotective molecules should be these without immunosuppressive activities. The present invention provides just such a method.

### SUMMARY OF THE INVENTION

The present invention is directed to a method of preventing and treating radical prostatectomy-induced eretion dysfuntion.

One embodiment of the present invention is directed to a method of preventing and treating radical prostatectomy-induced eretion dysfuntion by administering to a patient in need at least one thiosemicarbazone compound.

Another embodiment of the present invention is directed to a method of preventing and treating radical prostatectomy-induced eretion dysfuntion by administering to a patient in need a composition comprising 3-aminopyridine-2-carboxaldehyde thiosemicarbazone, or an analogue thereof.

Another embodiment of the present invention is directed to a method of preventing and treating radical prostatectomy-induced eretion dysfuntion by administering to a patient in need a composition comprising 3-aminopyridine-2-carboxaldehyde thiosemicarbazone. The step of administering is intravenous, intraperitoneal, subcutaneous, intramuscular, topical, transdermal, oral, or direct application to and surrounding cavernous nerves.

The present invention further encompasses methods of preventing and treating radical prostatectomy-induced eretion dysfuntion by administering a composition comprising a compound of Formula I, or an analogue thereof:

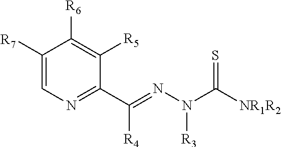

Wherein R, R₁, R₂, R₃, and R₄ are independently selected from the group consisting of hydrogen, C1-8alkyl, C2-8alkenyl, C2-8alkynyl, C3-8cycloalkyl, C1-8haloalkyl, C6-10aryl, amino-C1-8alkyl, hydroxy-C1-8alkyl, C1-8alkoxye-C1-8alkyl, and C1-8alkanoyl, or NR₁R₂ taken in combination form a 3 to 7 member ring which may comprise 0, 1, or 2 additional ring heteroatoms selected from N, O, and S; R₆ is hydrogen, hydroxy, amino, or C1-8alkyl; R₅ and R₇ are independently selected from the group consisting of hydrogen, halide, hydroxy, thiol, amino, hydroxyamino, mono-C1-8alkylamino, di(C1-8alkyl)amino, C1-8alkoxy, C1-8alkyl, C1-8alkenyl, and C2-8alkynyl.

The present invention further encompasses methods of preventing and treating radical prostatectomy-induced eretion dysfuntion by administering a composition comprising a compound of Formula II, or an analogue thereof:

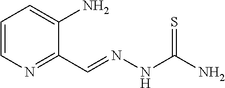 [SMILES: NC(=S)N/N=C/c1ncccc1N]

### DETAILED DESCRIPTION OF THE INVENTION

For simplicity and illustrative purposes, the principles of the present invention are described by referring to various exemplary embodiments thereof. Although the preferred embodiments of the invention are particularly disclosed herein, one of ordinary skill in the art will readily recognize that the same principles are equally applicable to, and can be implemented in other systems, and that any such variation would be within such modifications that do not part from the scope of the present invention. Before explaining the disclosed embodiments of the present invention in detail, it is to be understood that the invention is not limited in its application to the details of any particular arrangement shown, since the invention is capable of other embodiments. The terminology used herein is for the purpose of description and not of limitation. Further, although certain methods are described with reference to certain steps that are presented herein in certain order, in many instances, these steps may be performed in any order as would be appreciated by one skilled in the art, and the methods are not limited to the particular arrangement of steps disclosed herein.

The present invention is directed to a method for the preventing and treating radical prostatectomy-induced eretion dysfuntion comprising the step of administering to a patient a composition comprising a thiosemicarbazone compound. The means for synthesis of thiosemicarbazone compounds useful in the methods of the invention are well known in the art. Such synthetic schemes are described in U.S. Pat. Nos. 5,281,715; 5,767,134; 4,447,427; 5,869,676 and 5,721,259; all of which are incorporated herein by reference in their entirety.

The chemical structures of 3-aminopyridine-2-carboxaldehyde thiosemicarbazone analogues are shown in U.S. Pat. No 7,456,179, and patent applications of 20090275587, 20060194810 and 20060160826 each of which are hereby incorporated by reference.

The pharmaceutical compositions required by the present invention typically comprise a compound useful in the methods of the invention and a pharmaceutically acceptable carrier. As used herein “pharmaceutically acceptable carrier” includes any and all solvents, dispersion media, coatings, antibacterial and antifungal agents, isotonic and absorption delaying agents, and the like that are physiologically compatible. The type of carrier can be selected based upon the intended route of administration. In various embodiments, the carrier is suitable for intravenous, intraperitoneal, subcutaneous, intramuscular, topical, transdermal or oral administration, or direct application to and surrounding cavernous nerves. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, monostearate salts and gelatin. Moreover, the compounds can be administered in a time release formulation, for example in a composition which includes a slow release polymer. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are generally known to those skilled in the art.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof

Depending on the route of administration, the compound may be coated in a material to protect it from the action of enzymes, acids and other natural conditions which may inactivate the agent. For example, the compound can be administered to a subject in an appropriate carrier or diluent co-administered with enzyme inhibitors or in an appropriate carrier such as liposomes. Pharmaceutically acceptable diluents include saline and aqueous buffer solutions. Enzyme inhibitors include pancreatic trypsin inhibitor, diisopropylfluoro-phosphate (DEP) and trasylol. Liposomes include water-in-oil-in-water emulsions as well as conventional liposomes. Dispersions can also be prepared in glycerol, liquid polyethylene glycols, and mixtures thereof and in oils. Under ordinary conditions of storage and use, these preparations may contain a preservative to prevent the growth of microorganisms.

The active agent in the composition (i.e., one or more thiosemicarbazones) preferably is formulated in the composition in a therapeutically effective amount. A “therapeutically effective amount” refers to an amount effective, at dosages and for periods of time necessary, to achieve the desired therapeutic result to thereby influence the therapeutic course of a particular disease state. A therapeutically effective amount of an active agent may vary according to factors such as the disease state, age, sex, and weight of the individual, and the ability of the agent to elicit a desired response in the individual. Dosage regimens may be adjusted to provide the optimum therapeutic response. A therapeutically effective amount is also one in which any toxic or detrimental effects of the agent are outweighed by the therapeutically beneficial effects. In another embodiment, the active agent is formulated in the composition in a prophylactically effective amount. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

The amount of active compound in the composition may vary according to factors such as the disease state, age, sex, and weight of the individual. Dosage regimens may be adjusted to provide the optimum therapeutic response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

A compound of the invention can be formulated into a pharmaceutical composition wherein the compound is the only active agent therein. Alternatively, the pharmaceutical composition can contain additional active agents. For example, two or more compounds of the invention may be used in combination.

3-aminopyridine-2-carboxaldehyde thiosemicarbazone (hereinafter “PAN-811”), with a molecular weight of 195.24 Da, has demonstrated potent neuroprotective activities in several neurodegenerative models. PAN-811 was originally developed for cancer therapy due to its ability to inhibit ribonucleotide reductase, a key enzyme required for DNA synthesis. Previous studies demonstrated that PAN-811 at concentration of 0.45 μM fully blocked ischemic neurodegeneration and at 1.2μM completely halted hypoxia-induced neuronal cell death. PAN-811 was administered intracerebroventricularly (i.c.v.) at a dose of 50 μg per rat at 1 h after arterial occlusion. Staining of consecutive brain sections and computer-assisted quantitative analysis demonstrated that PAN-811 reduced the infarct volume by 59% in PAN-811 treated rats. The effect of a single intravenous (i. v.) bolus injection of PAN-811 was also investigated. Two-hour middle cerebral artery occlusion (MCAo) with cerebral blood flow reduction of 75% or greater resulted in infarct formation, brain edema and a significant number of premature deaths. PAN-811 treatment reduced infarct volume in a dose dependent manner with a maximal protection of 50% at a dose of 2mg/kg. PAN-811 treatment (2mg/kg) also resulted in a 70% reduction in brain edema volume. Accordingly, the mortality in PAN-811 treated groups was collectively reduced by 44%. Mechanistically PAN-811 prevents glutamate-induced excitatory cytotoxicity, veratridine-induced sodium channel opening and related cell death, and staurosporine-induced apoptosis. Nearly complete neuroprotection against glutamate insult is observed in cultured neuronal cells if the cells were pretreated with 10 μM PAN-811 for 24 h. In culture, ischemic condition results in a 19-fold increase in intracellular free calcium. PAN-811 at a dose of 5 μM reduced this elevated level by 72%. In a cell-free system by taking EDTA as a positive control, PAN-811 chelates free calcium as efficiently as EDTA. In addition, PAN-811 effectively suppresses oxidative stress in many ways. PAN-811 at a concentration as low as 1 μM suppressed in vitro hydrogen peroxide-induced LDH release by 78% (with P<0.01, compared to untreated/H₂O₂-insulted group) and at a concentration of 10 μM achieved maximal protection (by 90% comparing with untreated and H₂O₂-insulted group) with an EC₅₀ of ≠0.55 μM. PAN-811 also inhibited oxidative stress-induced cell death of human Alzheimer's disease-derived and age-matched olfactory neuroepithelial cells via suppression of intracellular reactive oxygen species. Importantly, PAN-811 manifested as a free radical scavenger in a cell free system where PAN-811 reduced 500 μM of a stable free radical diphenylpicrylhydrazyl by 70%. Taken together, PAN-811 has manifested as a potent neuroprotectant with dual drug targets—oxidative stress and free calcium.

Based on the key roles of excitoneurotoxicity and oxidative stress in radical prostatectomy-induced eretion dysfuntion and also the potent free calcium chelating and antioxidative effects of PAN-811, it has been discovered that PAN-811 is a preventing and therapeutic agent for radical prostatectomy-induced eretion dysfuntion. PAN-811 should not only inhibit radical prostatectomy-induced eretion dysfuntion, but also nerve-sparing radical prostatectomy.

While the invention has been described with reference to certain exemplary embodiments thereof, those skilled in the art may make various modifications to the described embodiments of the invention without departing from the scope of the invention. The terms and descriptions used herein are set forth by way of illustration only and not meant as limitations. In particular, although the present invention has been described by way of examples, a variety of compositions and processes would practice the inventive concepts described herein. Although the invention has been described and disclosed in various terms and certain embodiments, the scope of the invention is not intended to be, nor should it be deemed to be, limited thereby and such other modifications or embodiments as may be suggested by the teachings herein are particularly reserved, especially as they fall within the breadth and scope of the claims here appended. Those skilled in the art will recognize that these and other variations are possible within the scope of the invention as defined in the following claims and their equivalents.

What is claimed is:

1. A method for preventing and treating radical prostatectomy-induced erectile dysfuntion comprising the step of administering to a patient a composition comprising at least one thiosemicarbazone compound, or an analogue thereof.

2. The method of claim 1, wherein the at least one thiosemicarbazone compound comprises 3-aminopyridine-2-carboxaldehyde thiosemicarbazone (PAN-811).

3. The method of claim 2, wherein the step of administering is intravenous, intraperitoneal, subcutaneous, intramuscular, topical, transdermal, oral, or direct application to and surrounding cavernous nerves.

4. The method of claim 2, wherein the composition is an injectable and/or infusable solution.

5. The method of claim 2, wherein the composition is formulated as a micro emulsion.

6. The method of claim 2, wherein the composition is formulated as a liposome.

7. A method for the preventing and treating radical prostatectomy-induced eretion dysfuntion comprising administering to a patient a composition comprising at least one thiosemicarbazone compound (Formula I), or an analogue thereof:

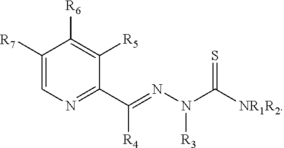

8. The method of claim 7, wherein the at least one thiosemicarbazone compound comprises the compound of Formula II, or an analogue thereof:

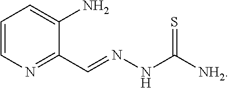 [SMILES: NC(=S)N/N=C/c1ncccc1N]

9. The method of claim 7, wherein the step of administering is intravenous, intraperitoneal, subcutaneous, intramuscular, topical, transdermal, oral, or direct application to and surrounding cavernous nerves.

10. The method of claim 7, wherein the composition is an injectable and/or infusible solution.

11. The method of claim 7, wherein the composition is formulated as a micro emulsion.

12. The method of claim 7, wherein the composition is formulated as a liposome.
